(19) United States
(12) Patent Application Publication
(10) Pub. No.: US 2004/0258646 A1
(43) Pub. Date: Dec. 23, 2004

(54) Oil-in-water emulsions containing ascorbic acid

(75) Inventors: Jens Nielsen, Henstedt-Ulzburg (DE); Thomas Raschke, Pinneberg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/818,832

(22) Filed: Apr. 5, 2004

Related U.S. Application Data
(63) Continuation of application No. PCT/EP02/10806, filed on Sep. 26, 2002.

(30) Foreign Application Priority Data
Oct. 4, 2001 (DE) 101 48 827.0

(51) Int. Cl. A61K 7/06; A61K 7/11

(52) U.S. Cl. 424/70.13

(57) ABSTRACT

The invention is an oil-in-water emulsion, comprising:

(a) one or more interface-active substances A selected from the group consisting of glucose derivatives which are characterized by the structural formula

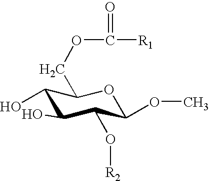

where R is a branched or unbranched alkyl radical having 1 to 24 carbon atoms, where R₁ and R₂, independently of one another, are either a hydrogen atom or a branched or unbranched alkyl radical having 1 to 24 carbon atoms,

(b) one or more interface-active substances B selected from the group consisting of substances of the general structural formula

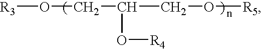

where R₃, R₄ and R₅, independently of one another, are selected from the group consisting of H and branched or unbranched, saturated or unsaturated fatty acid esters having 8 to 24 carbon atoms in which up to three aliphatic hydrogen atoms may be substituted by hydroxyl groups and n is a number from 2 to 8, and

(c) ascorbic acid.

The invention also includes methods of preparing emulsions using the emulsifiers (a) and (b) and methods of using the emulsion in cosmetic or dermatological compositions for treating skin conditions.

### CROSS-REFERENCE TO RELATED APPLICATIONS

This is a continuation application of PCT/EP02/10806, filed Sep. 26, 2002, which is incorporated herein by reference in its entirety, and also claims the benefit of German Priority Application No. 101 48 827.0, filed Oct. 4, 2001.

### FIELD OF THE INVENTION

The present invention relates to emulsion-based skincare products which comprise ascorbic acid as active ingredient.

### BACKGROUND OF THE INVENTION

Ascorbic acid is a highly effective and water-soluble skincare active ingredient which is characterized by its limited stability. The few known products available on the market which comprise more than 3% by weight of ascorbic acid are either formulations which comprise no water or little water, or they are W/O (water-in-oil) emulsions. These formulations have considerable sensory deficits compared with O/W (oil-in-water) emulsions. The use of more than 1% by weight of ascorbic acid in O/W emulsions has hitherto not been possible because ascorbic acid is not stable to increased electrolyte concentrations.

The specification WO 98/22075 describes cosmetic preparations containing ascorbic acid compounds, certain low-melting sugar or sugar alcohol carboxylates, and either certain alkylbenzoic alkyl esters or else certain isoalkylcarboxylic alkyl esters.

The specification WO 96/05797 describes cosmetic preparations which comprise 0.5-1% of ascorbic acid besides numerous other components.

The specification U.S. Pat. No. 6,024,942 describes photoprotective formulations comprising certain fractions of photoprotective agents, of hydrophobic constituents, hydrophilic surface-active substances, thickeners, water and a substance which lightens the skin, which may also be ascorbic acid.

The specification EP 771557 describes the use of ascorbic acid in a cosmetic method for the treatment of seborrhea.

The specification EP 779071 describes cosmetic and/or dermatological W/O/W emulsions with an aqueous phase with a water activity of at most 0.85, in which at least one water-sensitive active ingredient is present.

The specification DE 19650473 describes cosmetic preparations which comprise alkyl and/or alkenyl oligoglycosides, polyolpoly-12-hydroxystearate and dihydroxyacetone.

EP 666735 describes the use of agents which lighten the skin such as, for example, ascorbic acid in combination with certain partially etherified hydroquinones or hydroxyhydroquinones, and retinol derivatives for reducing or preventing skin damage caused by UV light, and/or lightening the skin.

The specification EP 839520 describes cosmetic and/or dermatological preparations in the form of transparent gels which comprise, inter alia, at least one polyol and at least one C₅₋₇-carbohydrate fatty acid ester.

The specification U.S. Pat. No. 6,024,942 describes photoprotective formulations which, besides photoprotective agents, surface-active substances, thickeners and water, also comprise an agent for lightening the skin, which may also be ascorbic acid.

The article “Topical Vitamin C in Aging” by R. M. Colven and S. R. Pinnell in the journal Clinics in Dermatology, Volume 14, pages 227-234 from 1996 describes the advantageous effect of ascorbic acid for controlling oxidative stress, in particular in connection with skin aging.

The article “The effects of topical L(+) lactic acid and ascorbic acid on skin whitening” by W. P. Smith, International Journal of Cosmetic Science, Volume 21, pages 33-40 from 1999 describes how ascorbic acid, in combination with lactic acid, has a pigmentation-lightening effect.

The article “Stabilization of ascorbic acid by microencapsulation in liposomes” by C. J. Kirby et al. in the International Journal of Food Science and Technology, Volume 26, pages 437-449 from 1991 describes how ascorbic acid can be stabilized by encapsulation in liposomes.

The article “Effect of stabilizers on the color stability of ascorbic acid cream determined by Tristimulus Colorimetry” by T.-C. Lin et al. in the Chinese Pharmaceutical Journal, Volume 48, pages 127-137 from 1996 describes the stabilization of ascorbic acid by dicarboxylic acids, amino acids, cyclodextrins and organic acids and salts thereof.

The article “Protective effect against sunburn of combined systemic ascorbic acid (vitamin C) and D-α-tocopherol (vitamin E)” by B. Eberlein-Konig et al in the Journal of the American Academy of Dermatology, Volume 38, pages 45-48 from 1998 describes an effect of vitamin C and vitamin E which moderates the appearance of sunburn.

The article “Regulation of Collagen Synthesis in Human Dermal Fibroblasts by the Sodium and Magnesium Salts of Ascorbyl-2 Phosphate” by J. C. Geesin et al. in Skin Pharmacology, Volume 6, pages 65-71 from 1993 describes the use of ascorbic 2-phosphate ester, which is more stable in water compared with free ascorbic acid, for stimulating collagen biosynthesis.

The article “Histopathological, morphometric and stereological studies of ascorbic acid and magnesium ascorbyl phosphate in a skin care formulation” by G. M. Silva and P. M. B. G. Maia Campos in the International Journal of Cosmetic Science, Volume 22, pages 169-179 from 2000 likewise describes the use of ascorbic acid derivatives, which are more stable in water compared with free ascorbic acid, for the stimulation of collagen biosynthesis in skincare formulations.

The article “Effects of Ascorbic Acid on Proliferation and Collagen Synthesis in Relation to the Donor Age of Human Dermal Fibroblasts” by C. L. Phillips et al. in the Journal of Investigative Dermatology, Volume 103, pages 228-232 from 1994 describes the stimulating effect of ascorbic acid on cell proliferation and collagen synthesis.

### SUMMARY OF THE INVENTION

It is evident from the present literature that there has been no lack of attempts to incorporate active ingredients which are sensitive toward water in a stable manner into cosmetic formulations, although this has hitherto not been possible in a satisfactory manner. Starting from this, the invention indicates a way of incorporating the water-sensitive active ingredient ascorbic acid in a stable, simple and bioavailable manner into O/W emulsions.

Entirely unforeseeably for the person skilled in the art, it has been found that the use of mixtures of

(a) one or more interface-active substances A chosen from the group of glucose derivatives which are characterized by the structural formula

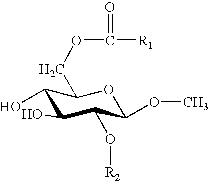

where R is a branched or unbranched alkyl radical having 1 to 24 carbon atoms, where R₁ and R₂, independently of one another, are either a hydrogen atom or a branched or unbranched alkyl radical having 1 to 24 carbon atoms,

(b) one or more interface-active substances B chosen from the group of substances of the general structural formula

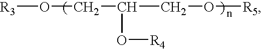

where R₃, R₄ and R₅, of one another, are chosen from the group which includes: H, branched or unbranched, saturated or unsaturated fatty acid esters having 8 to 24 carbon atoms in which up to three aliphatic hydrogen atoms may be substituted by hydroxyl groups and n is a number from 2 to 8 as emulsifier in O/W emulsions significantly improves the stability and the effectiveness of ascorbic acid in such formulations.

Mixtures of interface-active methylglucose distearate and triglyceryl dicarboxylates with the generic formula

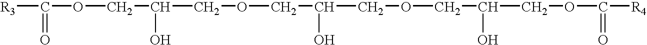

in which R₃ and R₄, independently of one another, are chosen from the group which includes: H, branched or unbranched, saturated or unsaturated fatty acid radicals having 14 to 20 carbon atoms are chosen can also preferably be used as emulsifiers.

Approximately equimolar mixtures of methylglucose distearate and triglyceryl dicarboxylate in which the radicals R₃ and R₄ are preferably both a stearate radical have proven particularly useful. Such emulsifier combinations are available as “polyglyceryl(3) methylglucose distearate” (PGMS) under the trade name Tego Care® 450 from Th. Goldschmidt KG.

In this connection, it is preferred that the content of ascorbic acid is 0.001-5.0% by weight, based on the total weight of the preparation.

Emulsions of this type can advantageously be used for producing cosmetic or dermatological preparations, it being particularly preferred to additionally incorporate antioxidants, which may very particularly preferably be ubiquinone (Q10) and/or α-glucosylrutin. Preparations of this type are particularly suitable for the treatment or prevention of UV light-induced skin damage and of skin-aging effects and also for the treatment of pigment disorders or for lightening the skin.

The invention also provides a process for the stable preparation of O/W emulsions containing electrolyte in particular containing ascorbic acid, characterized in that mixtures of

(a) one or more interface-active substances A chosen from the group of glucose derivatives which are characterized by the structural formula

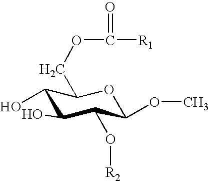

where R is a branched or unbranched alkyl radical having 1 to 24 carbon atoms, where R₁ and R₂, independently of one another, are either a hydrogen atom or a branched or unbranched alkyl radical having 1 to 24 carbon atoms,

(b) one or more interface-active substances B chosen from the group of substances of the general structural formula

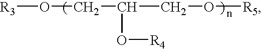

where R₃, R₄ and R₅, of one another, are chosen from the group which includes: H, branched or unbranched, saturated or unsaturated fatty acid esters having 8 to 24 carbon atoms in which up to three aliphatic hydrogen atoms may be substituted by hydroxyl groups and n is a number from 2 to 8, are used as emulsifier.

In this process, preference is given to using methylglucose distearate as interface-active substances A, and triglyceryl dicarboxylates of the generic formula

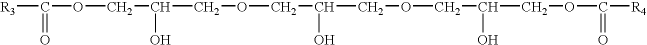

in which R₃ and R₄, independently of one another, are chosen from the group which includes: H, branched or unbranched, saturated or unsaturated fatty acid radicals having 14 to 20 carbon atoms, as interface-active substances B.

Approximately equimolar mixtures of methylglucose distearate and triglyceryl dicarboxylate in which the radicals R₃ and R₄ are preferably both a stearate radical have proven particularly useful. Such emulsifier combinations are available as “Polyglyceryl(3) methylglucose distearate” (PGMS) under the trade name Tego Care® 450 from Th. Goldschmidt KG.

It had therefore not been foreseen by the person skilled in the art that the emulsions used according to the invention or cosmetic or dermatological preparations comprising them would

be more effective as antioxidants,

be more effective as free-radical scavengers,

better prevent the binding of harmful photo products to lipids, DNA and proteins,

better stimulate the synthesis of collagen, hyaluronic acid and elastin, and increase cell renewal and regeneration of the skin,

better prevent pigment disorders, dry skin states and horny layer barrier disorders,

be more effective against skin aging,

better protect against wrinkles, age spots, and teleangiektases,

better protect the skin against photo reaction and photo induced skin damage, and

better prevent inflammatory reactions

than the preparations of the prior art. In addition, it had not been foreseen that the emulsions used according to the invention in cosmetic or dermatological preparations would have higher stability than the active ingredients used in each case individually, which applies particularly to the ascorbyl compounds and very particularly to vitamin C.

A particularly advantageous embodiment of the present invention is regarded as being the use of the described emulsions in the control or prophylaxis of oxidative stress.

### DETAILED DESCRIPTION OF PREFERRED EMBODIMENTS

The cosmetic or dermatological preparations according to the invention can have the customary composition and be used for the treatment, care and cleansing of the skin or hair and as a make-up product in decorative cosmetics. They preferably comprise 0.001% by weight to 10% by weight, preferably 0.05% by weight to 5% by weight, in particular 0.1 to 2.0% by weight, based on the total weight of the preparations, of active ingredient combinations used according to the invention.

It is preferred according to the invention to add complexing agents to the described emulsions.

Complexing agents are auxiliaries known per se in cosmetology and medicinal technology. By complexing undesirable metals, such as Mn, Fe, Cu and others, it is possible, for example, to prevent undesired chemical reactions in cosmetic or dermatological preparations.

Complexing agents, in particular chelating agents, form complexes with metal atoms; in the presence of one or more polybasic complexing agents, i.e. chelating agents, these complexes represent metallacycles. Chelating agents are compounds in which an individual ligand occupies more than one coordination site on a central atom. In this case, therefore, compounds which are normally extended are closed as a result of complexation via a metal atom or ion to give rings. The number of bonded ligands depends on the coordination number of the central metal. A prerequisite for chelate formation is that the compound which reacts with the metal contains two or more atomic groups which act as electron donors.

The complexing agent or agents can advantageously be chosen from the group of customary compounds, preference being given to at least one substance from the group consisting of tartaric acid and anions thereof, citric acid and anions thereof, aminopolycarboxylic acids and anions thereof (such as, for example, ethylenediamine-tetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, and trans-1,2-diaminocyclohexanetetraacetic acid (CDTA) and anions thereof).

According to the invention, the complexing agent or agents are advantageously present in cosmetic or dermatological preparations preferably in an amount of from 0.01% by weight to 10% by weight, preferably an amount of 0.05% by weight to 5% by weight, particularly preferably in an amount of 0.1 to 2.0% by weight, based on the total weight of the preparations.

For use, the cosmetic and dermatological preparations are, according to the invention, applied to the skin and/or hair in an adequate amount in the manner customary for cosmetics.

Cosmetic and dermatological preparations according to the invention may be present in various forms. Thus, for example, they may be a solution, an anhydrous preparation, an emulsion or microemulsion of the water-in-oil type (W/O) or a multiple emulsions, for example of the water-in-oil-in-water type (W/O/W), a gel, a solid stick, an ointment and also an aerosol.

The cosmetic and dermatological preparations according to the invention can comprise cosmetic auxiliaries, as are customarily used in such preparations, e.g. preservatives, bactericides, perfumes, antifoams, dyes, pigments which have a coloring effect, thickeners, surface-active substances, emulsifiers, softening, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

For the purposes of the present invention, emulsions according to the invention are, for example, creams, lotions, cosmetic milk preparations, and mousse cream, which can be dispensed from an aerosol container. These comprise, for example, fats, oils, waxes or other fatty substances, and also water and one or more emulsifiers, as are customarily used for such a type of formulation.

In particular, emulsions used according to the invention can also be combined with other antioxidants or free-radical scavengers.

Such antioxidants are advantageously chosen from the group consisting of amino acids (for example glycine, histidine, tyrosine, and tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, and lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoeyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine-sulfoximines, homocysteine-sulfoximine, buthionine sulfones, penta-, hexa- and heptathionine-sulfoximine) in very low tolerated doses (for example pmol to μmol/kg), and furthermore (metal) chelating agents (for example α-hydroxy fatty acids, palmitic acid, phytic acid, and lactoferrin), α-hydroxy acids (for example citric acid, lactic acid, and malic acid), humic acid, bile acid, bile extracts, bilirubin, biliverdin, EDTA, EGTA and derivatives thereof, unsaturated fatty acids and derivatives thereof (for example γ-linolenic acid, linoleic acid, and oleic acid), folic acid and derivatives thereof, ubiquinone and ubiquinol and derivatives thereof, tocopherols and derivatives (for example vitamin E acetate), vitamin A and derivatives (vitamin A palmitate) and coniferyl benzoate of benzoin resin, rutinic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, sesamol, sesamolin, zinc and derivatives thereof (for example ZnO and ZnSO₄), selenium and derivatives thereof (for example selenomethionine), stilbenes and derivatives thereof (for example stilbene oxide and trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.05-20% by weight, in particular 0.1 to 10% by weight, based on the total weight of the preparation.

If vitamin E or derivatives thereof are the additional antioxidant(s), it is advantageous to choose their respective concentrations from the range 0.001-10% by weight, based on the total weight of the formulation.

If vitamin A or vitamin A derivatives, or carotenes or derivatives thereof are the additional antioxidant(s), it is advantageous to choose their respective concentrations from the range from 0.001-10% by weight, based on the total weight of the formulation.

Emulsions according to the invention are advantageous and comprise, for example, said fats, oils, waxes and other fatty substances, and also water and an emulsifier, as is customarily used for such a type of formulation.

The lipid phase can advantageously be chosen from the following group of substances:

mineral oils and mineral waxes;

oils, such as triglycerides of capric or of caprylic acid, and also natural oils, such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates; and

silicone oils, such as dimethylpolysiloxanes, diethylpolysiloxanes, diphenylpolysiloxanes, and mixed forms thereof.

The oil phase of the emulsions, oleogels, hydrodispersions or lipodispersions for the purposes of the present invention is advantageously chosen from the group of esters of saturated or unsaturated, branched or unbranched alkanecarboxylic acids having a chain length of from 3 to 30 carbon atoms and saturated or unsaturated, branched or unbranched alcohols having a chain length of from 3 to 30 carbon atoms, from the group of esters of aromatic carboxylic acids and saturated or unsaturated, branched or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated or unsaturated, branched or unbranched alkanecarboxylic acids having a chain length of from 8 to 24 carbon atoms, in particular 12-18 carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, and dicaprylyl ether.

Particularly advantageous mixtures are those of C₁₂₋₁₅-alkyl benzoate and 2-ethyl-hexyl isostearate, those of C₁₂₋₁₅-alkyl benzoate and isotridecyl isononanoate, and those of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

Advantageously, the oil phase can also have a content of cyclic or linear silicone oils, or consist entirely of such oils, although it is preferred to use an additional content of other oil phase components apart from the silicone oil or the silicone oils.

Cyclomethicone (octamethylcyclotetrasiloxane) is advantageously used as the silicone oil to be used according to the invention. However, other silicone oils can also be used advantageously for the purposes of the present invention, for example hexa-methylcyclotrisiloxane, polydimethylsiloxane, and poly(methylphenylsiloxane).

Mixtures of cyclomethicone and isotridecyl isononanoate, and of cyclomethicone and 2-ethylhexyl isostearate are also particularly advantageous.

Very particularly preferred oil components are octyldodecanol, caprylic/capric triglyceride, cyclomethicone, C₁₂-C₁₅ alkyl benzoate and dicaprylyl carbonate.

In addition to the alkyl glycoside, preparations according to the invention present in the form of emulsions may comprise one or more further emulsifiers. These emulsifiers may advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearates, sorbitan stearates, glyceryl stearyl citrates and sucrose stearates),

b) ethoxylated fatty alcohols and fatty acids,

c) ethoxylated fatty amines, fatty acid amides, and fatty acid alkanolamides,

d) alkylphenol polyglycol ethers (e.g. Triton X), and

e) sugar derivatives (esters and/or ethers of glucose, sucrose and other sugars).

The anionic emulsifiers include

a) soaps (e.g. sodium stearate),

b) fatty alcohol sulfates, and

c) mono-, di-trialkyl phosphoric esters and ethoxylates thereof.

The cationic emulsifiers include

a) quaternary ammonium compounds with a long-chain aliphatic radical, e.g. distearyldiammonium chloride.

The amphoteric emulsifiers include

a) alkylaminoalkanecarboxylic acid,

b) betaines and sulfobetaines, and

c) imidazoline derivatives

In addition, there are naturally occurring emulsifiers to which beeswax, wool wax, lecithin and stearols belong.

O/W emulsifiers can, for example, advantageously be chosen from the group of polyethoxylated, polypropoxylated or polyethoxylated and polypropoxylated products, e.g.:

fatty alcohol ethoxylates,

ethoxylated wool wax alcohols,

polyethylene glycol ethers of the general formula R—O—(—CH₂—CH₂—O—)ₙ—R′,

fatty acid ethoxylates of the general formula R—COO—(—CH₂—CH₂—O—)ₙ—H,

etherified fatty acid ethoxylates of the general formula

R—COO—(—CH₂—CH₂—O—)ₙ—R′,

esterified fatty acid ethoxylates of the general formula

R—COO—(—CH₂—CH₂—O—)ₙ—C(O)—R′,

polyethylene glycol glycerol fatty acid esters,

ethoxylated sorbitan esters,

cholesterol ethoxylates,

ethoxylated triglycerides,

alkyl ether carboxylic acids of the general formula

R—O—(—CH₂—CH₂—O—)ₙ—CH₂—COOH

wherein n is a number from 5 to 30,

polyoxyethylene sorbitol fatty acid esters,

alkyl ether sulfates of the general formula R—O—(—CH₂—CH₂—O—)ₙ—SO₃—H,

fatty alcohol propoxylates of the general formula R—O—(—CH₂—CH(CH₃)—O—)ₙ—H,

polypropylene glycol ethers of the general formula

R—O—(—CH₂—CH(CH₃)—O—)ₙ—R′,

propoxylated wool wax alcohols,

etherified fatty acid propoxylates R—COO—(—CH₂—CH(CH₃)—O—)ₙ—R′,

esterified fatty acid propoxylates of the general formula

R—COO—(—CH₂—CH(CH₃)—O—)ₙ—C(O)—R′,

fatty acid propoxylates of the general formula R—COO—(—CH₂—CH(CH₃)—O—)ₙ—H,

polypropylene glycol glycerol fatty acid esters,

propoxylated sorbitan esters,

cholesterol propoxylates,

propoxylated triglycerides,

alkyl ether carboxylic acids of the general formula

R—O—(—CH₂—CH(CH₃)O—)ₙ—CH₂—COOH,

alkyl ether sulfates or the parent acids of these sulfates of the general formula

R—O—(—CH₂—CH(CH₃)—O—)ₙ—SO₃—H,

fatty alcohol ethoxylates/propoxylates of the general formula R—O—Xₙ—Yₘ—H,

polypropylene glycol ethers of the general formula R—O—Xₙ—Yₘ—R′,

etherified fatty acid propoxylates of the general formula R—COO—Xₙ—Yₘ—R′, and

fatty acid ethoxylates/propoxylates of the general formula R—COO—Xₙ—Yₘ—H.

According to the invention, the polyethoxylated, polypropoxylated or polyethoxylated and polypropoxylated O/W emulsifiers are particularly advantageously chosen from the group of substances with HLB values of 11-18, very particularly advantageously with HLB values of 14.5-15.5, if the O/W emulsifiers have saturated radicals R and R′. If the O/W emulsifiers have unsaturated radicals R or R′, or if isoalkyl derivatives are present, then the preferred HLB value of such emulsifiers may also be lower or higher.

It is advantageous to choose the fatty alcohol ethoxylates from the group of ethoxylated stearyl alcohols, cetyl alcohols, and cetylstearyl alcohols (cetearyl alcohols). Particular preference is given to polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group: polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, and polyethylene glycol(20) oleate.

Sodium laureth-11 carboxylate can advantageously be used as the ethoxylated alkyl ether carboxylic acid or salt thereof.

Sodium laureth-14 sulfate can advantageously be used as alkyl ether sulfate.

Polyethylene glycol(30) cholesteryl ether can advantageously be used as an ethoxylated cholesterol derivative. Polyethylene glycol(25) soyastearol has also proven useful.

The polyethylene glycol(60) evening primrose glycerides can advantageously be used as ethoxylated triglycerides.

In addition, it is advantageous to choose the polyethylene glycol glyceryl fatty acid esters from the group consisting of polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, and polyethylene glycol(18) glyceryl oleate/cocoate.

It is likewise favorable to choose the sorbitan esters from the group consisting of polyethylene glycol(20) sorbitan monolaurate, polyethylene glycol(20) sorbitan monostearate, polyethylene glycol(20) sorbitan monoisostearate, polyethylene glycol(20) sorbitan monopalmitate, and polyethylene glycol(20) sorbitan monooleate.

Advantageous W/O emulsifiers which may be used are: fatty alcohols having 8 to 30 carbon atoms, monoglycerol esters of saturated or unsaturated, branched or unbranched alkanecarboxylic acids with a chain length of from 8 to 24, in particular 12-18, carbon atoms, diglycerol esters of saturated or unsaturated, branched or unbranched alkanecarboxylic acids with a chain length of from 8 to 24, in particular 12-18, carbon atoms, monoglycerol ethers of saturated or unsaturated, branched or unbranched alcohols with a chain length of from 8 to 24, in particular 12-18, carbon atoms, diglycerol ethers of saturated or unsaturated, branched or unbranched alcohols with a chain length of from 8 to 24, in particular 12-18, carbon atoms, propylene glycol esters of saturated or unsaturated, branched or unbranched alkanecarboxylic acids with a chain length of from 8 to 24, in particular 12-18, carbon atoms, and sorbitan esters of saturated or unsaturated, branched or unbranched alkanecarboxylic acids with a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monoolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate, and glyceryl monocaprylate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ethers, propylene glycol monomethyl, monoethyl or monobutyl ethers, diethylene glycol monomethyl or monoethyl ethers and analogous products, and also alcohols of low carbon number, e.g. ethanol, isopropanol, 1,2-propanediol, glycerol, and in particular, one or more thickeners which may advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example carbopol grades 980, 981, 1382, 2984, 5984, ETD 2001, ETD 2020, ETD 2050, Ultrez 10, in each case individually or in combination.

In particular, mixtures of the abovementioned solvents are used. In the case of alcoholic solvents, water may be a further constituent.

Emulsions according to the invention are advantageous and comprise, for example, said fats, oils, waxes and other fatty substances, and also water and an emulsifier, as is customarily used for such a type of formulation.

Suitable propellants for preparations according to the invention which can be sprayed from aerosol containers are the customary known readily volatile, liquefied propellants, for example hydrocarbons (propane, butane and isobutane), which can be used on their own or in a mixture with one another. Compressed air can also be used advantageously.

The preparations according to the invention can advantageously further comprise substances which absorb UV radiation in the UVB region, the total amount of the filter substances being, for example, 0.1% by weight to 30% by weight, preferably 0.5 to 10% by weight, in particular 1.0 to 6% by weight, based on the total weight of the preparations, in order to make available cosmetic preparations which protect the hair or the skin against the entire range of ultraviolet radiation. They can also serve as sunscreens for the hair or the skin.

If the preparations according to the invention comprise UVB filter substances, these may be oil-soluble or water-soluble. Examples of oil-soluble UVB filters which are advantageous according to the invention are:

3-benzylidenecamphor derivatives, preferably 3-(4-methylbenzylidene)camphor and 3-benzylidenecamphor;

4-aminobenzoic acid derivatives, preferably 2-ethylhexyl 4-(dimethyl-amino)benzoate and amyl 4-(dimethylamino)benzoate;

esters of cinnamic acid, preferably 2-ethylhexyl 4-methoxycinnamate and isopentyl 4-methoxycinnamate;

esters of salicylic acid, preferably 2-ethylhexyl salicylate, 4-isopropylbenzyl salicylate and homomenthyl salicylate,

derivatives of benzophenone, preferably 2-hydroxy-4-methoxybenzophenone, 2-hydroxy-4-methoxy-4′-methylbenzophenone and 2,2′-dihydroxy-4-methoxy-benzophenone; and

esters of benzylmalonic acid, preferably di(2-ethylhexyl) 4-methoxybenzal-malonate-2,4,6-trianilino(p-carbo-2′-ethyl-1′-hexyloxy)-1,3,5-triazine.

Examples of advantageous water-soluble UVB filters are:

salts of 2-phenylbenzimidazole-5-sulfonic acid, such as its sodium, potassium or its triethanolammonium salt, and the sulfonic acid itself;

sulfonic acid derivatives of benzophenones, preferably 2-hydroxy-4-methoxy-benzophenone-5-sulfonic acid and its salts; and

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts, and 1,4-di(2-oxo-10-sulfo-3-bornylidenemethyl)benzene and salts thereof (the corresponding 10-sulfato compounds, for example the corresponding sodium, potassium or triethanol-ammonium salt), also referred to as benzene-1,4-di(2-oxo-3-bornylidenemethyl-10-sulfonic acid.

The list of specified UVB filters which can be used in combination with the active ingredient combinations according to the invention is not of course intended to be limiting.

The invention also provides for the use of a combination of the emulsions used according to the invention with at least one UVB filter as antioxidant, and for the use of a combination of the active ingredient combinations used according to the invention with at least one UVB filter as antioxidant in a cosmetic or dermatological preparation.

It may also be advantageous to use UVA filters which are customarily present in cosmetic preparations. These substances are preferably derivatives of dibenzoyl-methane, in particular 1-(4′-tert-butylphenyl)-3-(4′-methoxyphenyl)propane-1,3-dione and 1-phenyl-3-(4′-isopropylphenyl)propane-1,3-dione.

In addition, advantageous UVA filters originate from the group of triazines, such as, for example, 2,4-bis{[4-(2-ethylhexyloxy)-2-hydroxy]phenyl}-6-(4-methoxyphenyl)-1,3,5-triazine (trade name Tinosorb® S), and the group of triazoles, such as, for example, 2,2′-methylenebis[6-2H-benzotriazole-2-yl]-4-(1,1,3,3-tetramethylbutyl)phenol (trade name Tinosorb® M). An advantageous water-soluble UVA filter is the sodium salt of 2′-bis(1,4-phenylene)-1H-benzimidazole4,6-disulfonic acid (trade name Neo Heliopan AP®). It is possible to use the amounts used for the UVB combination.

The invention also provides for the use of a combination of emulsions used according to the invention with at least one UVA filter as antioxidant, and for the use of a combination of the active ingredient combinations according to the invention with at least one UVA filter as antioxidant in a cosmetic or dermatological preparation.

The invention also provides for the use of a combination of emulsions according to the invention with at least one UVA filter and/or at least one UVB filter as an antioxidant in a cosmetic or dermatological preparation.

Emulsions according to the invention may also comprise inorganic pigments which are customarily used in cosmetics for protecting the skin against UV rays. These are oxides of titanium, zinc, zirconium, silicon, manganese, cerium and mixtures thereof, and also modifications in which the oxides are the active agents. Particular preference is given to pigments based on titanium dioxide.

These combinations of UVA filters and pigment or preparations which comprise this combination are also provided by the invention. The amounts given for the above combinations can be used.

Cosmetic and dermatological preparations for protecting the hair against UV rays according to the invention are, for example, shampoos, preparations which are applied during rinsing of the hair before or after shampooing, before or after permanent wave treatment, before or after dyeing or bleaching of the hair, preparations for blow-drying or arranging the hair, preparations for coloring or bleaching, styling and treatment lotion, hair lacquer or permanent wave compositions.

The emulsions according to the invention comprise active ingredients and auxiliaries as are customarily used for this type of preparation for hair care and hair treatment. Auxiliaries include preservatives, surface-active substances, antifoams, thickeners, emulsifiers, fats, oils, waxes, organic solvents, bactericides, perfumes, dyes or pigments whose task is to color the hair or the cosmetic or dermatological preparation itself, electrolytes and substances to combat hair greasiness.

For the purposes of the present invention, electrolytes are understood as meaning water-soluble alkali metal, ammonium, alkaline earth metal (including magnesium) and zinc salts of inorganic anions and any mixtures of such salts, it being necessary to ensure that these salts are pharmaceutically or cosmetically safe.

The anions according to the invention are preferably chosen from the group of chlorides; sulfates and hydrogensulfates; phosphates, hydrogenphosphates and linear and cyclic oligophosphates; and carbonates and hydrogencarbonates.

Cosmetic preparations which are in the form of a skin cleanser or shampoo preferably comprise at least one anionic, nonionic or amphoteric surface-active substance, or else mixtures of such substances, the active ingredient combinations used according to the invention in the aqueous medium and auxiliaries as are customarily used therefor. The surface-active substance or the mixtures of these substances can be present in the shampoo in a concentration between 1% by weight and 50% by weight.

If the cosmetic or dermatological preparations are in the form of a lotion which is rinsed out and applied, for example, before or after bleaching, before or after shampooing, between two shampooing steps, before or after permanent wave treatment, they are, for example, aqueous or aqueous-alcoholic solutions which optionally comprise surface-active substances whose concentration may be between 0.1 and 10% by weight, preferably between 0.2 and 5% by weight.

The cosmetic or dermatological preparations may also be aerosols containing the auxiliaries customarily used therefor.

A cosmetic preparation in the form of a lotion which is not rinsed out, in particular a lotion for arranging the hair, a lotion which is used during the blow-drying of hair, a styling and treatment lotion, is generally an aqueous, alcoholic or aqueous-alcoholic solution and comprises at least one cationic, anionic, nonionic or amphoteric polymer or else mixtures thereof, and active ingredient combinations used according to the invention in an effective concentration. The amount of the polymers used is, for example, between 0.1 and 10% by weight, preferably between 0.1 and 3% by weight.

Cosmetic preparations for the treatment and care of the hair which comprise the active ingredient combinations used according to the invention may be in the form of emulsions which are of the nonionic or anionic type. Besides water, nonionic emulsions comprise oils or fatty alcohols which may, for example, also be polyethoxylated or polypropoxylated, or else mixtures of the two organic components. These emulsions optionally comprise cationic surface-active substances.

According to the invention, cosmetic preparations for the treatment and care of the hair can be in the form of gels which, in addition to an effective content of isoquercitrin and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The present invention also covers a cosmetic method of protecting the skin and the hair against oxidative and/or photooxidative processes which is characterized in that a cosmetic composition which comprises emulsions according to the invention is applied to the skin or hair in a sufficient amount.

Although the cosmetic or dermatological preparations achieved according to the invention and the active ingredient combinations present therein are characterized by increased stability toward oxidative influence, storage forms are nevertheless preferred in which the ingress of atmospheric oxygen is reduced. Thus, for example, bottling under inert gas, in particular nitrogen, is advantageous. Advantageous packaging has proven to be, in particular, aluminum tubes, glass jars, plastic tubes, plastic-aluminum laminate tubes, airless dispensers and aluminum aerosol cans.

It is of course known to the person skilled in the art that high-quality cosmetic preparations are in most cases inconceivable without customary auxiliaries and additives. These include, for example, consistency-imparting agents, fillers, perfume, additional active ingredients, such as vitamins or proteins, insect repellants, alcohol, water, salts, antimicrobial proteolytic or keratolytic substances etc.

### EXAMPLES

The examples below are intended to illustrate the invention without limiting it.

Unless stated otherwise, all amounts, percentages or parts refer to the weight, in particular to the total weight of the preparations or of the particular mixtures.

| | Example Number | | | |
|---|---|---|---|---|
| | 1 | 2 | 3 | 4 |
| Polyglyceryl-3 methylglucose distearate | 3 | 2 | 3 | 5 |
| Sorbitan stearate | | | 2 | |
| Stearyl alcohol | | | | 2 |
| Cetearyl alcohol | | 5 | 2 | |
| Cetyl alcohol | 4 | | | 3 |
| Myristyl myristate | | 1 | | |
| C₁₂₋₁₅ alkyl benzoate | 2 | 2 | | 1 |
| Caprylic/capric triglycerides | | | 4 | 5 |
| Ethylhexyl coconut fatty acid ester | 3 | 5 | | |
| Octyldodecanol | | | 3 | 4 |
| Vaseline | 4 | | | |
| Octamethyltetrasiloxane (cyclomethicone) | 4 | 5 | 3 | |
| Dicaprylyl carbonate | | | | 2 |
| Polydecene | | 1 | | |
| PEG-150 distearate | | 1 | | |
| TiO₂ | | 1 | 1 | |
| Ethylhexyl methoxycinnamate | 3 | 2 | | |
| 2-ethylhexyl 2-cyano-3-diphenylacrylate (octocrylene) | | | 3 | 5 |
| Ethylhexyltriazone | | 2 | | |
| Butylmethoxydibenzoylmethane | | 1 | 1 | |
| Bis-ethylhexyloxyphenolmethoxyphenoltriazines | 1 | | | |
| Ubiquinone (Q10) | 0.03 | | | 0.05 |
| Tocopheryl acetate | | 0.5 | | |
| α-Glucosylrutin | 0.1 | | | |
| Ascorbic acid | 3 | 1 | 3 | 3 |
| Trisodium EDTA | 0.2 | | 0.2 | 0.2 |
| Iminodisuccinate | | 0.2 | | |
| Phenoxyethanol | 0.3 | | 0.3 | 0.5 |
| p-Hydroxybenzoic alkyl esters (Paraben) | 0.5 | 0.4 | 0.3 | 0.4 |
| Hexamidine diisethionate | 0.2 | | | |
| 1,3-dimethylol-5,5-dimethylhydantoin (DMDM hydantoin) | | 0.2 | | |
| Diazolidinylurea | | | 0.1 | |
| Ethanol, denatured | | 2 | | |
| Xanthan gum | | 0.1 | 0.2 | 0.2 |
| Polyacrylic acid (carbomer) | 0.5 | 0.3 | 0.1 | |
| Polyacrylamide | | | | 0.1 |
| Glycerol | 8 | 10 | 6 | 10 |
| Panthenol | 2 | 1 | 1 | |
| Natural oils, plant extracts (Hamamelis, sunflower oil) | 0.3 | 0.1 | | |
| Fillers (distarch phosphate, SiO₂, talc) | 5 | 4 | | 1 |
| Perfume | q.s. | q.s. | q.s. | q.s. |
| Water | Ad | Ad | Ad | Ad |
| | 100 | 100 | 100 | 100 |
| | Example Number | | | |
| | 5 | 6 | 7 | 8 |
| Polyglyceryl-3 methylglucose distearate | 2 | 4 | 4 | 5 |
| Sorbitan stearate | 1 | | 2 | |
| Stearyl alcohol | | | | 1 |
| Cetearyl alcohol | | 2 | 2 | 4 |
| Cetyl alcohol | 3 | | | |
| C₁₂₋₁₅ alkyl benzoate | 2 | 2 | | 1 |
| Caprylic/capric triglycerides | | | 2 | 5 |
| Ethylhexyl coconut fatty acid ester | 3 | 5 | | |
| Octyldodecanol | | | | 4 |
| Vaseline | 4 | | | |
| Octamethyltetrasiloxane (cyclomethicone) | 4 | 5 | 4 | |
| Polydecene | | 1 | 2 | |
| Dicaprylyl carbonate | 1 | | 3 | |
| TiO₂ | 1 | | 2 | |
| Ethylhexyl methoxycinnamate | 2 | 2 | | |
| 2-ethylhexyl 2-cyano-3-diphenylacrylate (octocrylene) | | | | 5 |
| Ethylhexyltriazone | | 2 | 3 | |
| Butylmethoxydibenzoylmethane | | 1 | | 1 |
| Bis-ethylhexyloxyphenolmethoxyphenyltriazines | 1 | | | |
| Ubiquinone (Q10) | 0.03 | | | 0.04 |
| Tocopheryl acetate | | 1.0 | | |
| α-Glucosylrutin | | | 0.1 | |
| Ascorbic acid | 3 | 2 | 3 | 4 |
| Trisodium EDTA | 0.2 | | | 0.2 |
| Iminodisuccinate | | 0.2 | 0.2 | |
| Phenoxyethanol | 0.3 | | 0.3 | 0.2 |
| p-Hydroxybenzoic alkyl ester (Paraben) | 0.5 | 0.4 | 0.5 | 0.4 |
| Hexamidine diisethionate | 0.2 | | | |
| 1,3-dimethylol-5,5-dimethylhydantoin (DMDM hydantoin) | | 0.2 | | |
| Diazolidinylurea | | | | 0.1 |
| Ethanol, denatured | | 2 | | |
| Xanthan gum | | 0.1 | 0.2 | 0.1 |
| Polyacrylic acid (carbomer) | 0.3 | 0.3 | 0.1 | |
| Polyacrylamide | | | | |
| Glycerol | 8 | 10 | 6 | 8 |
| Panthenol | | 1 | 1 | |
| Natural oils, plant extracts (Hamamelis, sunflower oil) | | 0.1 | | 0.2 |
| Fillers (distarch phosphate, SiO₂, talc) | | 3 | | 1 |
| Perfume | q.s. | q.s. | q.s. | q.s. |
| Water | Ad | Ad | Ad | Ad |
| | 100 | 100 | 100 | 100 |

That which is claimed:

1. An oil-in-water emulsion, comprising:
(a) one or more interface-active substances A selected from the group consisting of glucose derivatives which are characterized by the structural formula 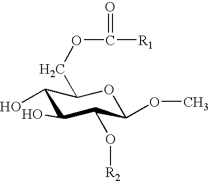
where R is a branched or unbranched alkyl radical having 1 to 24 carbon atoms, where R₁ and R₂, independently of one another, are either a hydrogen atom or a branched or unbranched alkyl radical having 1 to 24 carbon atoms,
(b) one or more interface-active substances B selected from the group consisting of substances of the general structural formula 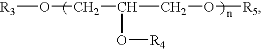
where R₃, R₄ and R₅, independently of one another, are selected from the group consisting of H and branched or unbranched, saturated or unsaturated fatty acid esters having 8 to 24 carbon atoms in which up to three aliphatic hydrogen atoms may be substituted by hydroxyl groups and n is a number from 2 to 8, and
(c) ascorbic acid.

2. The emulsion as claimed in claim 1, wherein the one or more interface-active substances A include methylglucose distearate.

3. The emulsion as claimed in claim 1, wherein the one or more interface-active substances B include triglyceryl dicarboxylates with the generic formula 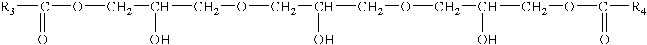
in which R₃ and R₄, independently of one another, are selected from the group consisting of H and branched or unbranched, saturated or unsaturated fatty acid radicals having 14 to 20 carbon atoms.

4. The emulsion as claimed in claim 1, wherein the one or more interface-active substances A include methylglucose distearate, and the one or more interface-active substances B include triglyceryl dicarboxylates with the generic formula 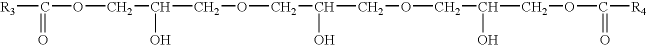
in which R₃ and R₄, independently of one another, are selected from the group consisting of H and branched or unbranched, saturated or unsaturated fatty acid radicals having 14 to 20 carbon atoms.

5. The emulsion as claimed in claim 4, comprising an approximately equimolar mixture of the methylglucose distearate and the triglyceryl dicarboxylates.

6. The emulsion as claimed in claim 5, wherein the triglyceryl dicarboxylates have the generic formula 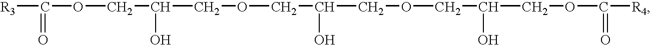
wherein R₃ and R₄ are both stearate radicals.

7. The emulsion as claimed in claim 4, wherein the triglyceryl dicarboxylates have the generic formula 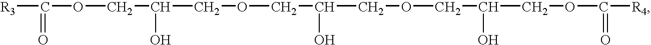
wherein R₃ and R₄ are both stearate radicals.

8. The emulsion as claimed in claim 1, wherein the ascorbic acid is present in an amount of 0.001-5.0% by weight, based on the total weight of the emulsion.

9. The emulsion as claimed in claim 1, wherein the ascorbic acid is present in an amount of 1.0-5.0% by weight, based on the total weight of the emulsion.

10. The emulsion as claimed in claim 1, wherein the ascorbic acid is present in an amount of 3.0-5.0% by weight, based on the total weight of the emulsion.

11. A cosmetic or dermatological preparation, comprising the emulsion of claim 1.

12. The cosmetic or dermatological preparation as claimed in claim 11, further comprising an antioxidant.

13. The cosmetic or dermatological preparation as claimed in claim 12, wherein the antioxidant comprises one or more of ubiquinone (Q10) and α-glucosylrutin.

14. A cosmetic or dermatological preparation, comprising the emulsion of claim 4.

15. A process for preparing a stable oil-in-water emulsion, comprising combining:
(a) one or more interface-active substances A selected from the group consisting of glucose derivatives which are characterized by the structural formula 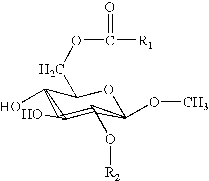
where R is a branched or unbranched alkyl radical having 1 to 24 carbon atoms, where R₁ and R₂, independently of one another, are either a hydrogen atom or a branched or unbranched alkyl radical having 1 to 24 carbon atoms,
(b) one or more interface-active substances B selected from the group consisting of substances of the general-structural formula 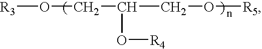
where R₃, R₄ and R₅, independently of one another, are selected from the group consisting of H and branched or unbranched, saturated or unsaturated fatty acid esters having 8 to 24 carbon atoms in which up to three aliphatic hydrogen atoms may be substituted by hydroxyl groups and n is a number from 2 to 8, and
(c) an electrolyte.

16. The process as claimed in claim 15, wherein the electrolyte is ascorbic acid.

17. The process as claimed in claim 15, wherein the one or more interface-active substances A include methylglucose distearate, and the one or more interface-active substances B include triglyceryl dicarboxylates with the generic formula 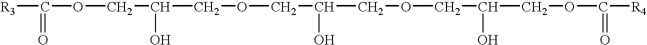
in which R₃ and R₄, independently of one another, are selected from the group consisting of H and branched or unbranched, saturated or unsaturated fatty acid radicals having 14 to 20 carbon atoms.

18. A method for one or more of treating and preventing one or more of UV light-induced skin damage and skin aging phenomena, comprising applying to the skin a cosmetic or dermatological preparation, comprising:
(a) one or more interface-active substances A selected from the group consisting of glucose derivatives which are characterized by the structural formula 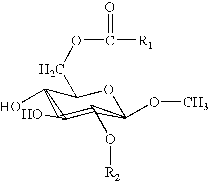
where R is a branched or unbranched alkyl radical having 1 to 24 carbon atoms, where R₁ and R₂, independently of one another, are either a hydrogen atom or a branched or unbranched alkyl radical having 1 to 24 carbon atoms,
(b) one or more interface-active substances B selected from the group consisting of substances of the general structural formula 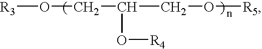
where R₃, R₄ and R₅, independently of one another, are selected from the group consisting of H and branched or unbranched, saturated or unsaturated fatty acid esters having 8 to 24 carbon atoms in which up to three aliphatic hydrogen atoms may be substituted by hydroxyl groups and n is a number from 2 to 8,
(c) ascorbic acid, and
(d) one or more antioxidants.

19. The process as claimed in claim 18, wherein the one or more interface-active substances A include methylglucose distearate, and the one or more interface-active substances B include triglyceryl dicarboxylates with the generic formula 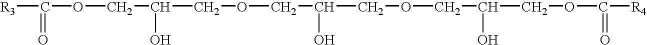
in which R₃ and R₄, independently of one another, are selected from the group consisting of H and branched or unbranched, saturated or unsaturated fatty acid radicals having 14 to 20 carbon atoms.

20. The process as claimed in claim 18, wherein the antioxidant comprises one or more of ubiquinone (Q10) and α-glucosylrutin.

21. A method for one or more of treating pigment disorders and lightening skin, comprising applying to the skin a cosmetic or dermatological preparation, comprising:
(a) one or more interface-active substances A selected from the group consisting of glucose derivatives which are characterized by the structural formula 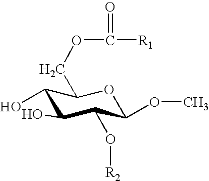
where R is a branched or unbranched alkyl radical having 1 to 24 carbon atoms, where R₁ and R₂, independently of one another, are either a hydrogen atom or a branched or unbranched alkyl radical having 1 to 24 carbon atoms,
(b) one or more interface-active substances B selected from the group consisting of substances of the general structural formula 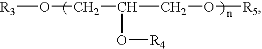
where R₃, R₄ and R₅, independently of one another, are selected from the group consisting of H and branched or unbranched, saturated or unsaturated fatty acid esters having 8 to 24 carbon atoms in which up to three aliphatic hydrogen atoms may be substituted by hydroxyl groups and n is a number from 2 to 8,
(c) ascorbic acid, and
(d) one or more antioxidants.

22. The process as claimed in claim 21, wherein the one or more interface-active substances A include methylglucose distearate, and the one or more interface-active substances B include triglyceryl dicarboxylates with the generic formula 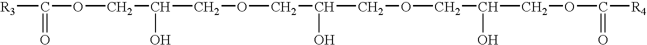
in which R₃ and R₄, independently of one another, are selected from the group consisting of H and branched or unbranched, saturated or unsaturated fatty acid radicals having 14 to 20 carbon atoms.

23. The process as claimed in claim 21, wherein the antioxidant comprises one or more of ubiquinone (Q10) and α-glucosylrutin.
